(19) United States
(12) Patent Application Publication
(10) Pub. No.: US 2005/0282765 A1
(43) Pub. Date: Dec. 22, 2005

(54) Enzyme having S-adenosyl-L-homocysteine hydrolase (AHCY) type activity

(75) Inventor: Derek N.J. Hart, Queensland (AU)

Correspondence Address: NIXON & VANDERHYE, PC, 901 NORTH GLEBE ROAD, 11TH FLOOR, ARLINGTON, VA, 22203, US

(73) Assignee: The Corporation of the Trustees of the Order of the Sisters of Mercy in Queensland, Queensland (AU)

(21) Appl. No.: 11/110,748

(22) Filed: Apr. 21, 2005

Related U.S. Application Data
(63) Continuation of application No. 09/782,051, filed on Feb. 14, 2001, now abandoned.
Application No. 09/782,051 is a continuation of application No. 09/269,906, filed on Apr. 29, 1999, now abandoned.

(30) Foreign Application Priority Data
Oct. 4, 1996 (NZ) 299507
Oct. 6, 1997 (WO) PCT/NZ97/00133

(51) Int. Cl. C12Q 1/68; C07H 21/04; A61K 48/00; C12N 9/88

(52) U.S. Cl. 514/44; 435/232; 435/69.1; 435/320.1; 435/325; 536/23.2; 530/388.26; 435/6

(57) ABSTRACT

Enzyme having AHCY-type activity and DNA sequences which encode the enzyme. The enzyme is useful in a screening assay to identify compounds useful as immunosuppression agents.

This invention relates to a novel enzyme of the S-adenosyl-L-homocysteine hydrolase type and to methods which employ this enzyme.

### BACKGROUND

S-adenosyl-L-homocysteine hydrolase (AHCY) is an enzyme which is involved in the pathway of S-adenosylmethionine metabolism. S-adenosylmethionine (AdoMet) plays a pivotal role as a methyl donor in a myriad of biological and biochemical events. For example. AdoMet donates methyl groups to small molecules like norepinephrine, yielding the potent product epinephrine: to medium sized molecules such as phosphatidyl ethanolamine, eventually forming phosphatidylcholine in the lipid bilayer; and to RNA and DNA, thereby modulating transcription and translation processes. AdoMet—dependent methylation reactions have been postulated to regulate biological phenomena by post-translational modification as seen in the carboxymethylation of proteins and perhaps membrane functions via methylation of membrane phospholipids¹⁶.

S-adenosyl homocysteine (AdoHcy), formed after donation of the methyl group of AdoMet to a methyl acceptor is then hydrolysed to adenosine (Ado) and homocysteine (Hcy) by AdoHcy hydrolase (AHCY). Ado can either be denigrated by Ado deaminase (ADA) to inosine or be phosphorylated by Ado deaminase to become AMP.

Inhibition of AHCY results in an increase in intracellular levels of AdoHcy which is a potent inhibitor of AdoMet dependent methylation reactions²⁻⁴. AdoHcy has also been reported to be a competitive inhibitor of PI Kinases⁵. PI kinase is required for PI metabolism and is involved in receptor indicated increases in intracellular calcium and the transduction of biochemical signals through cell membranes.

The applicants have now identified a novel enzyme of the AHCY type. It is towards this enzyme. DNA which encodes it, and methods which employ it that the present invention is inter alia broadly directed.

### SUMMARY OF THE INVENTION

Therefore, in a first aspect, the present invention provides an enzyme having AHCY-type activity which includes amino acids 177 to 314 of the amino acid sequence of FIG. 1, or a functional portion or functional equivalent of said enzyme.

Conveniently, the enzyme comprises amino acids 183 to 614 or 1 to 614 of the amino acid sequence of FIG. 1.

In a further aspect, the present invention, provides a DNA sequence which encodes the AHCY-type enzyme above, said sequence selected from the group of:
(a) a sequence which encodes an enzyme as defined above or a functional portion or equivalent thereof;
(b) a sequence which is a complement of a sequence (a);
(c) a sequence which is a reverse complement of a sequence (a); and
(d) a sequence which is a reverse sequence of a sequence (a).

Preferably, sequence (a) comprises nucleotides 529 to 945 of the FIG. 1 sequence, nucleotides 549 to 1844 of the FIG. 1 sequence, or nucleotides 1 to 1844 of the FIG. 1 sequence.

In yet a further aspect, the invention provides DNA constructs which comprise a DNA sequence as defined above.

In still a further aspect, the invention provides a method of modulating the activity of an AHCY-type enzyme as defined above in a patient which comprises administering to said patient a DNA construct as defined above.

In yet a further aspect, the invention provides a method of determining the modulatory potential of a test substance (particularly a compound), which method comprises the step of determining the ability of said substance to modulate the activity of the AHCY-type enzyme defined above.

Most conveniently, the modulatory potential which will be tested for will be the ability of the test substance to inhibit the AHCY-type enzyme defined above. In this way, immunosuppressant substances can be identified.

In still further aspects, the invention provides antibodies specific for the enzyme of the invention and optionally labelled nucleic acid probes (based upon the DNA sequence defined above). Such antibodies and probes have utility in the identification of abnormal FD cells in cytological preparations and tissue sections, and by in situ hybridisation, respectively.

### DESCRIPTION OF THE DRAWINGS

Although the present invention is broadly as described above, it will be appreciated that it also includes embodiments described below. In particular, a person skilled in the art will appreciate that the invention will be better understood by reference to the accompanying drawing in which:

FIG. 1 shows the cDNA and translated putative amino acid sequence of DD4b5.3. There is an open reading frame which extends without a stop codon for the full 5′ nucleotides sequence. The initiation codon has yet to be identified;

FIG. 2 shows the alignment of DD4b5.3 AHCY domain with the full length AHCY amino acid sequences of human (hm). mouse (mu) and drosophila (dr). Some conserved features shown to be important for AHCY function are indicated;

FIG. 3 provides a summary of the features of the DD4b5.3 sequence;

FIG. 4 shows the results of RT-PCR analysis of DD4b5.3 mRNA expression in different leucocyte populations. A Southern blot of the products probed with a specific internal probe is shown;

FIG. 5 shows the results of RT-PCR analysis of different purified DC populations to examine their expression of DD4b5.3 mRNA. A semiquantitative analysis (ethidium stain) at different cycle number is shown;

FIG. 6 shows the metabolic cycle of AdoMet and Ado Hcy: and

FIG. 7 summarises the RAP-PCR protocol.

### DESCRIPTION OF THE INVENTION

As a part of the their general investigations into dendritic cells, the applicants have identified a gene encoding an enzyme of the AHCY-type. The sequence of the identified gene has been found to have common features wits the gene sequence of AHCY itself.

The applicants have also determined that this gene has a restricted expression pattern, being found in fresh blood dendritic cells and cultured blood dendritic cells, but nor in T-cell or B-cell lymphocytes. It is therefore believed that the enzyme this gene encodes is substantially dendritic-cell-restricted. Due to the central role dendritic cells play in initiation of the primary immune response to foreign antigen, manipulation of the activity of this enzyme within dendritic cells (especially inhibition) will have a direct effect on the immune response of the host. This represents the primary, although not sole, application of this invention.

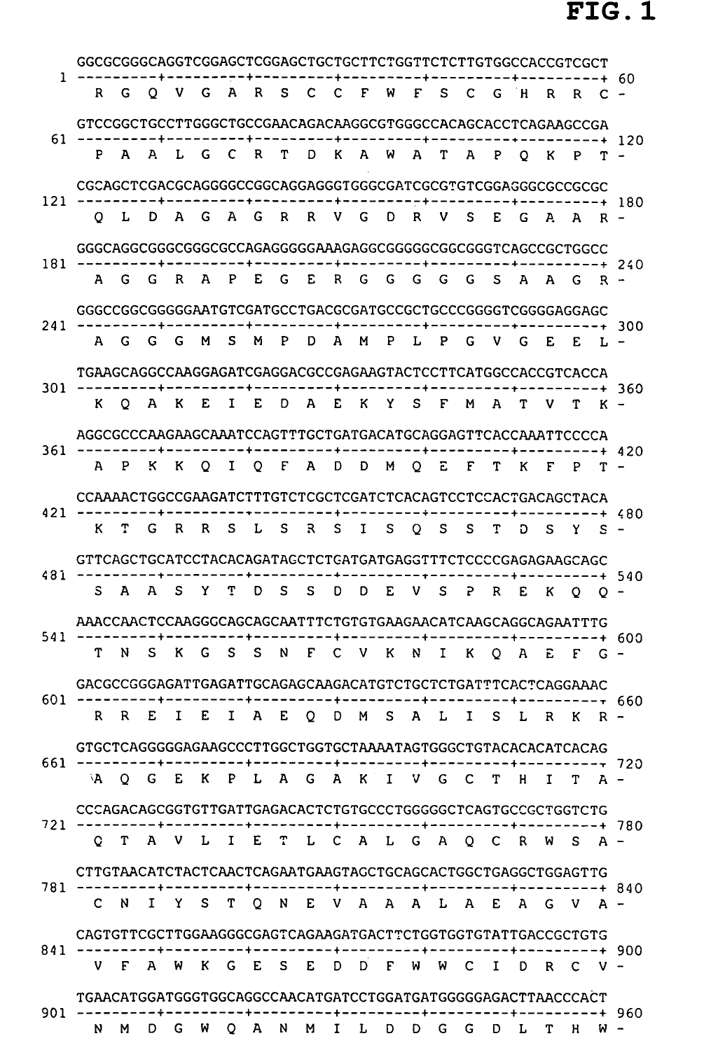

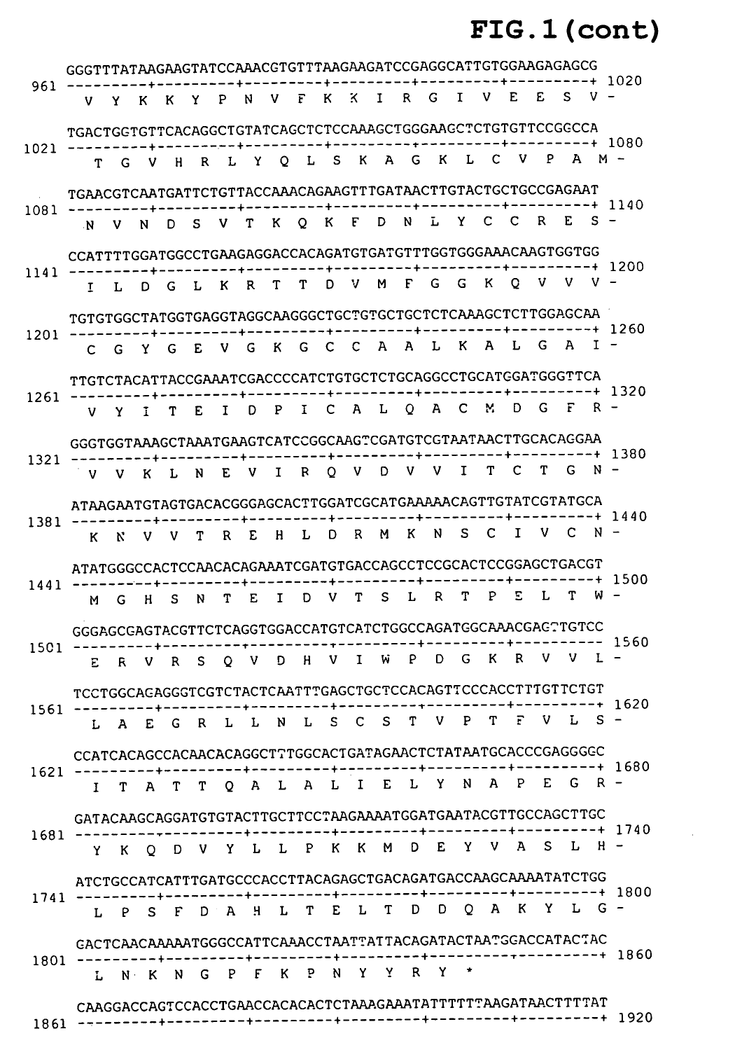

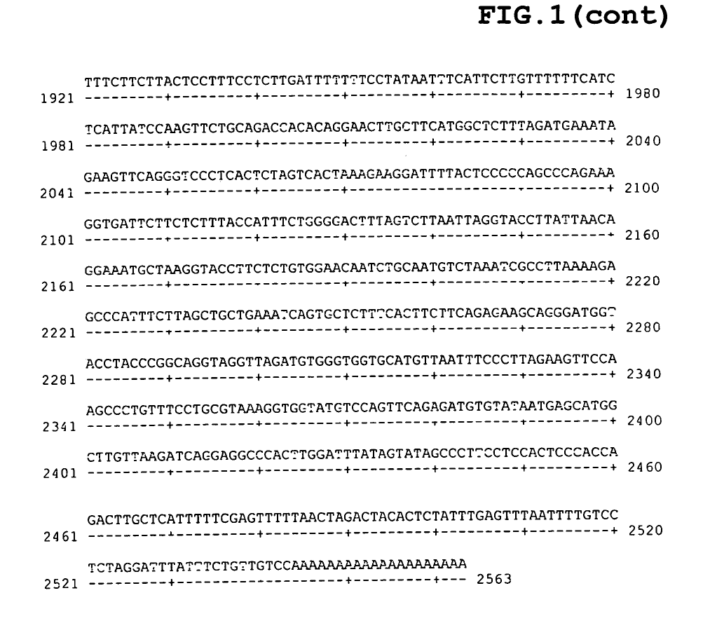

In its first aspect, the invention therefore provides the enzyme itself. As indicated above, the enzyme of the invention has AHCY-type activity and includes amino acids 177 to 314 of the amino acid sequence of FIG. 1 more preferably amino acids 183 to 614 of the amino acid sequence of FIG. 1, and most preferably amino acids 1 to 614 of the amino acid sequence of FIG. 1.

The Invention also contemplates functional portions of the enzyme. As used herein, the “functional portion” of an enzyme is that portion which contains the active site essential for affecting the metabolic step. i.e. the portion of the molecule that is capable of binding one or more recants or is capable of improving or regulating the rate of reaction. The active site may be made up of separate portions present on one or more polypeptide chains and will generally exhibit high substrate specificity.

The invention also includes functional equivalents of the enzyme described above.

The enzyme is a protein. A protein is considered a functional equivalent of another protein for a specific function if the equivalent protein is immunologically cross-reactive with, and has the same function as, the original protein. The equivalent may, for example, be a fragment of the protein, or a substitution, addition or deletion mutant of the protein.

For example, it is possible to substitute amino acids in a sequence with equivalent amino acids using conventional techniques. Groups of amino acids known normally to be equivalent are:

| | |
|---|---|
| (a) | Ala(A) Ser(S) Thr(T) Pro(P) Gly(G); |
| (b) | Asn(N) Asp(D) Glu(E) Gln(Q); |
| (c) | His(H) Arg(R) Lys(K); |
| (d) | Met(M) Leu(L) Ile(I) Val(V); |
| and | |
| (e) | Phe(F) Tyr(Y) Trp(W). |

Substitutions, additions and/or deletions in the enzyme may be made as long as the resulting equivalent enzyme is immunologically cross-reactive with, and has essentially the same function as, the native enzyme.

The equivalent enzymes will normally have substantially the same amino acid sequence as the native enzyme. An amino acid sequence that is substantially the same as another sequence, but that differs from the other sequence by means of one or more substitutions, additions and/or deletions is considered to be an equivalent sequence. Preferably, less than 25%, more preferably less than 10%, and most preferably less than 5% of the number of amino acid residues in the amino acid sequence of the native enzyme are substituted for, added to, or deleted from.

The enzyme of the invention will most conveniently be produced using recombinant techniques. Therefore, in a further embodiment, the present invention provides isolated complete or partial DNA sequences encoding, or partially encoding the enzyme of the invention. Specifically, the present invention provides isolated DNA sequences comprising nucleotides 529 to 945: 549 to 1844; or 1 to 1844 of the FIG. 1 sequence. Complements of such isolated DNA sequences, reverse complements of such isolated DNA sequences and reverse sequences of such isolated DNA sequences-together with variants of such sequences, are also provided. DNA sequences encompassed by the present invention include cDNA, genomic DNA, recombinant DNA and wholly or partially chemically synthesized DNA molecules.

The definition of the terms “complement”, “reverse complement” and “reverse secquence”, as used herein, is best illustrated by the following example. For the sequence 5′ AGGACC 3′, the compliment, reverse complement and reverse sequence are as follows:

| | |
|---|---|
| complement | 3′ TCCTGG 5′ |
| reverse complement | 3′ GGTCCT 5′ |
| reverse sequence | 5′ CCAGGA 3′. |

As used herein the term “variant” covers vary sequence which exhibits at least about 70% and, more preferably, at least about 90% identity to a sequence of the present invention. Most preferably, a “variant” is any sequence which has at least about a 99% probability of being the same as the inventive sequence. The probability for DNA sequences is measured by the computer algorithm FASTA (version 2.0u4. February 1996; Pearson W. R. et al., Proc. Natl. Acad. Sci., 85:2444-248 1988).

The DNA sequences may be isolated by high throughput sequencing of cDNA libraries. Alternatively, oligonucleotide probes based on the sequences provided in SEQ ID NOS. 4-6 can be synthesized and used to identify positive clones in either cDNA or genomic DNA libraries by means of hybridization or PCR techniques. Probes should be at least about 10. preferably at least about 15 and most preferably at least about 20 nucleotides in length. Hybridization and PCR techniques suitable for use with such oligonucleotide probes are well known in the art. Positive clones may be analyzed by restriction enzyme digestion. DNA sequencing or the like.

In addition, the DNA sequences of the present invention may be generated by synthetic means using techniques well known in the art. Equipment for automated synthesis of oligonucleotides is commercially available from suppliers such as Perkin Elmer/Applied Biosystems Division (Foster City, Calif.) and may be operated according to the manufacturers instructions.

The invention also provides DNA constructs. In one embodiment, the DNA constructs of the present invention include an open reading frame coding for at least a functional portion of the enzyme of the present invention.

For applications where amplification of enzyme activity is desired, the open reading frame is inserted in the DNA construct in a sense orientation, such that transformation with the DNA construct will lead to at. Increase in the number of copies of the gene and therefore an increase in the amount of enzyme. When down-regulation of activity is desired, the open reading frame is inserted in the DNA construct in an antisense orientation, such that the RNA produced by transcription of the DNA sequence is complementary to the endogenous mRNA sequence. This, in turn, wall result in a decrease in the number of copies of the gene and therefore a decrease in the amount of enzyme. Alternatively, regulation can be achieved by inserting appropriate sequences or subsequences (e.g. DNA or RNA, in ribozyme constructs.

In a second embodiment, the DNA constructs comprise a nucleotide sequence including a non-coding region of a gene coding for the enzyme of the present invention, or a nucleotide sequence complementary to such a non-coding region. As used herein the term, “non-coding region” includes both transcribed sequences which are not translated. Band non-transcribed sequences within about 2000 base pairs 5′ or 3′ of the translated sequences or open reading frames. Examples of non-coding regions which may be usefully employed in the inventive constructs include introns and 5′-non-coding leader sequences. Transformation with such a DNA construct may lead to a reduction in the amount of enzyme synthesized by the process of cosuppression.

The DNA constructs of the present invention further comprise a gene promoter sequence and a gene termination sequence, operably linked to the DNA sequence to be transcribed, which control expression of the gene. The gene promoter sequence is generally positioned at the 5′ end of the DNA sequence to be transcribed, and is employed to initiate transcription of the DNA sequence. Gene promoter sequences are generally found in the 5′ non-coding region of a gene but they may exist in introns (Luehrsen, K. R., Mol. Gen. Genet. 225:81-93, 1991) or in the coding region. When the construct includes an open reading frame in a sense orientation, the gene promoter sequence also initiates translation of the open reading frame. For DNA constructs comprising either an open reading frame in an antisense orientation or a non-coding-region, the gene promoter sequence consists only of a transcription initiation site having a RNA polymerase binding site.

A variety of gene promoter sequences which may be usefully employed in the DNA constructs of the present invention are well known in the art. The promoter gene sequence, and also the gene termination sequence, may be endogenous or may be exogenous, provided the promoter is functional in the host. Preferably, gene promote. Band termination sequences are from the inventive sequences themselves.

The gene termination sequence, which is located 3′ to the DNA sequence to be transcribed, may come from the same gene as the gene promoter sequence or may be from a different gene. Many gene termination sequences known in the art may be usefully employed in the present invention. However, preferred gene terminator sequences are those from tie original enzyme gene.

Techniques for operatively linking the components of the DNA constructs are well known in the art and include the use of synthetic linkers containing one or more restriction endonuclease sites as described, for example, by Maniatis et al., (Molecular Cloning: A Laboratory Manual, Cold Spring Harbor Laboratories. Cold Spring Harbor, N.Y., 1989). The DNA construct of the present invention may be linked to a vector having at least one replication system, for example, E. coli, whereby after each manipulation, the resulting construct can be cloned and sequenced and the correctness of the manipulation determined.

Having described the enzyme and the DNA constructs the present invention will now be illustrated by reference to the following non-limiting examples.

### EXAMPLE 1

(a) Identification of Enzyme-Encoding Gene

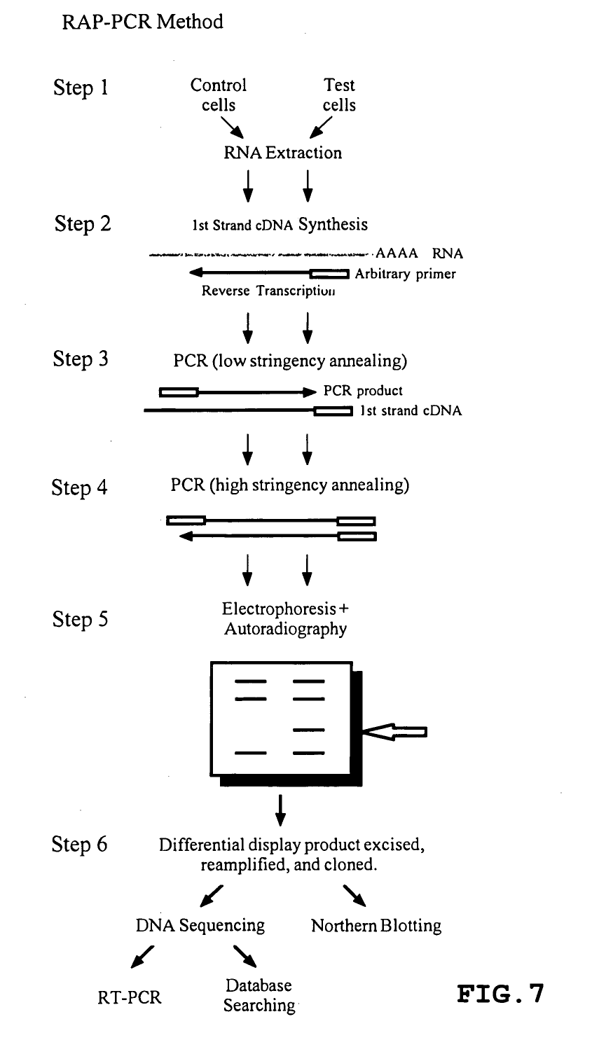

The Differential Display (DD) technique RAP-PCR was employed, using Hodgkins cell line L428 and moonocytoid cell line U937. The RAP-PCR protocol is as shown in FIG. 7, and was performed as follows:

Total RNA extracted from L428 and U937 cell lines (Step 1) was used for first stand cDNA was synthesis using an arbitrary oligonucleotide primer (Step 2). An aliquot of the RT reaction was used in a subsequent PCR with the same arbitrary primer in the presence of 32P-labelled dCTP. The first five cycles of the PCR was performed at the low annealing temperature of 40° C. (low stringency) to allow mismatches between the arbitrary primer and cDNA template (Step 3). followed by 30 cycles at 60° C. annealing temperature (high stringency) to amplify specifically the products which have incorporated the arbitrary primer (Step 4). The PCR products were electrophoresed non-denaturing sequencing gels and subjected to autoradiography. PCR products on the L428 lanes but not U937 lanes were excised from the gel using the autoradiogram as a guide (Step 5). and used as template to reamplify the products with the arbitrary primer (Step 6). Reamplified L428 specific bands were cloned in pBluescript and DNA sequenced.

One clone, called DD4b.5.3. contained a 416 bp fragment encoding part of the enzyme of the invention.

(b) Isolation of a Full Length cDNA for DD4b5.3

The DD4b5.3 cDNA fragment was used to probe a Lambda LAS express (Stratagene) L428 cDNA library produced in this laboratory. A near full length clone of 2.2 kb was identified (6.1922(1)) which was sequenced using a LI-COR automated sequencer (FIG. 1, nucleotides 241-2563).

Rapid amplification of cDNA ends (RACE) was used to identify further 5′ cDNA sequence using L428 cDNA as a template. RACE products were cloned and sequenced revealing further 5′ sequence. Subsequently a 2nd cDNA clone (2.11(1)b) was isolated from the L-428 library. This provided further 5′ sequence (FIG. 1; nucleotides 1-240) with two bases of identical overlapping sequence with the first clone.

Comparison of the nucleic acid sequence revealed a significant portion of identity with the cDNA sequence for an enzyme called L-adenosine-S homocysteine hydrolase (AHCY).

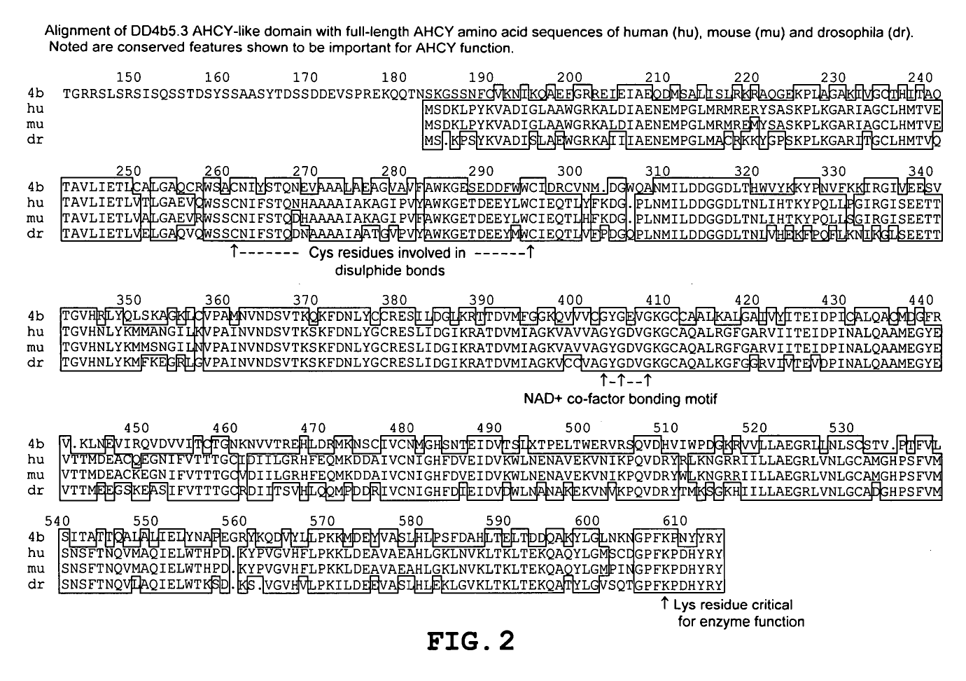

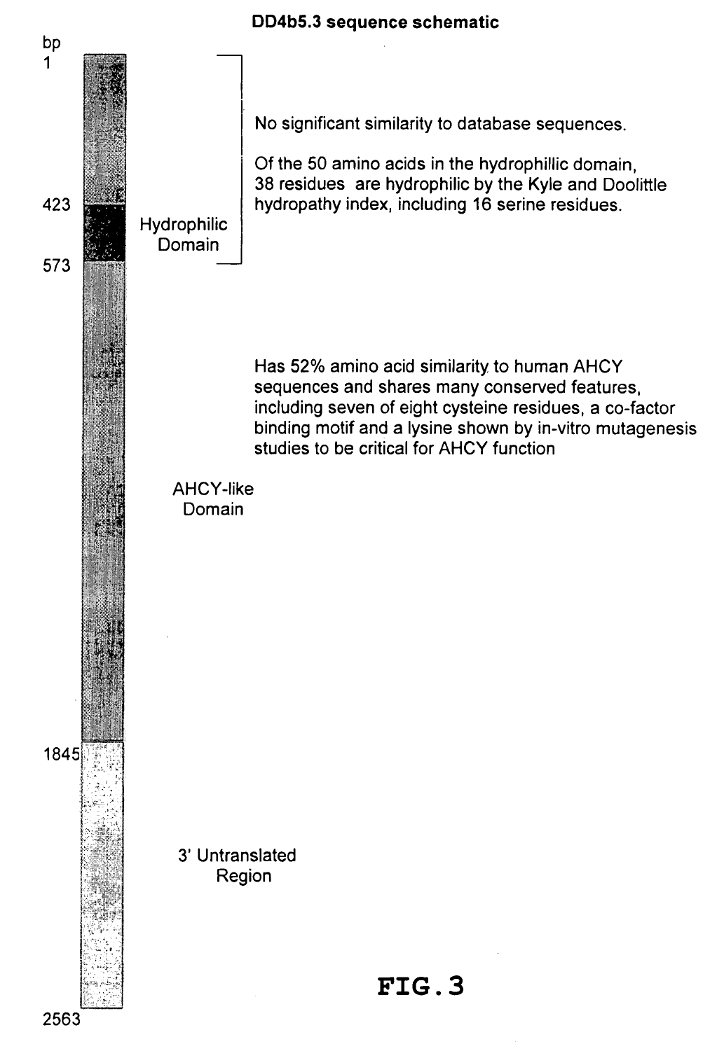

Translation of the cDNA sequence for DD4b5.3 revealed an open reading frame as indicated in FIG. 1. Comparison of the putative DD4b5.3 amino acid sequence with the AHCY sequences of human¹, mouse (Genbank Accession no. L32836) and drosophila (Genbank Accession no. X95636) revealed expensive similarity to the AHCY sequences. (FIG. 2). A further analysis of the structure is shown in FIG. 3.

DD4b5.3 has a similar enzymatic function to AHCY based on significant identities in the AHCY related sequence. Thus DD4b5.3 has:
i) Cys¹¹³ and Cys¹⁹⁵ are conserved (numbering based on AHCY amino acid sequence (DD4b5.3 sequence)). Modification of these affects enzymatic function¹³. In contrast Cys⁴²¹ is non essential¹³ and this is substituted (Lys) in DD4b5.3.
ii) Lys⁴²⁶ identified as critical for AHCY function is preserved in DD4b5.3¹⁴, (FIG. 2)
iii) Val¹⁷⁵ and Lys¹⁸⁶ predicted to be involved in Ade ring binding are conserved in DD4b5.3¹⁵. In contrast, Val³¹⁹ and Arg³²⁷ also predicted to be involved¹⁵ are not but the QVD sequence between them is, suggesting this may allow for an alternative specificity.
iv) Glu^{197 16} and Serine¹⁹⁸ which are also related to the active enzyme site are conserved in DD4b5.3.
v) The cofactor NAD binding site¹⁷ is highly conserved (Lys²¹⁴-Asp²³⁵) and the critical G-G—G binding site is present in DD4b5.3. (FIG. 2)
vi) Eight Cys residues are thought to be involved in disulphide bonding. These contribute to maintaining the globular structure of the 4 subunits (˜47,000) which make up the tetramer (˜190,000) AHCY molecule in mammals. Seven of eight are preserved in DD4b5.3 amino acid sequence.

### EXAMPLE 2

### Tissue Distribution

### Experimental 1

### Methods

Northern Blotting

Total RNA was extracted from p428 and U937 cell lines, electrophoresed on formaldehyde-denaturing agarose gels, transferred to nylon membranes and probed with 32P labeled DD clones.

RT-PCR Analysis

Based on sequence data, clone-specific oligonucleotide primers were synthesised to assess tissue distribution by RT-PCR. Two panels of haemopoetic cell populations were screened:
1) Cultured cell lines. Total RNA was extracted from cell lines, treated with PNAse-free DNAse I, and used for first stand cDNA synthesis. Aliquots of cDNA were used in PCRs with specific oligonucleotide primers.
2) Freshly isolated cells. First strand cDNA synthesis was performed directly on whole-cells isolated from human peripheral blood, based on established methods. Template consisted of 500 purified cells and the entire reaction was used in the subsequent PCR.

RT-PCR products were assessed by agarose gel electrophoresis. Identity of the products was confirmed by internal oligonucleotide hybridisation.

### Results

Northern Blot Analysis

Northern blot analysis reveals DD4b.5.3 probe hybridises only to the L428 lanes.

RT-PCR Analysis of Cell Lines

Because L428 is a Hodgkin's disease cell line it is possible that DD products isolated from L428 may be expressed in other. Hodgkin's cell lines. We assessed the expression of clone DD4b.5.3 in a cell line panel by RT-PCR (Table 1). The results indicate clone DD4b.5.3 is expressed showing a different pattern of expression across the different cell lines.

TABLE 1

| Cell line | DD4b.53 |
|---|---|
| L428 | +++ |
| act.U937 | + |
| U937 | + |
| Monomac | + |
| THP1 | ++ |
| NB4 | + |
| KG1 | − |
| HDLM2 | +++ |
| KMH2 | ++ |
| Raji | ++ |
| Mann | ++ |
| EBV-B cells | + |
| Jurkat | ++ |
| K562 | − |

Relative band intensities: +++ strong, ++ moderate, + weak, − no bank visible. β2-microglobulin cDNA control PCR also performed.

RT-PCR Analysis of Fresh Cells

To assay the expression of the differential display clones in normal cell populations. RT-PCR was performed on freshly isolated cells. The results are summarised in Table 2.

TABLE 2
Summary of RT-PCR results

| RT-PCR Template | Phenotype | DD4b.5.3 |
|---|---|---|
| Fresh blood DC | DR⁻, Mix | + |
| Cultured Blood DC | DR⁻, CMRF44 | ++ |
| Fresh Blood Monocytes | CD14⁻ | − |
| Cultured Blood Monocytes | CD14⁻ | − |
| Fresh B Blood Lymphocytes | CD19⁻ | − |
| Cultured B Blood Lymphocytes | CD19⁻ | − |
| Blood T Lymphocytes | CD3⁻ | − |
| NK cells | CD16⁻, CD57⁻ | − |

1 Semi-Nested PCR

Relative band intensities compared by ethidium bromide staning: ++ moderate, + weak, +/− very weak, − no product visible.

### Experimental 2

i) Cell Lines

Northern blot analysis using RNA obtained from cell lines revealed specific hybridisation to a L428 transcript which was not detected in U937 (data not shown)

ii) Leucocyte Populations

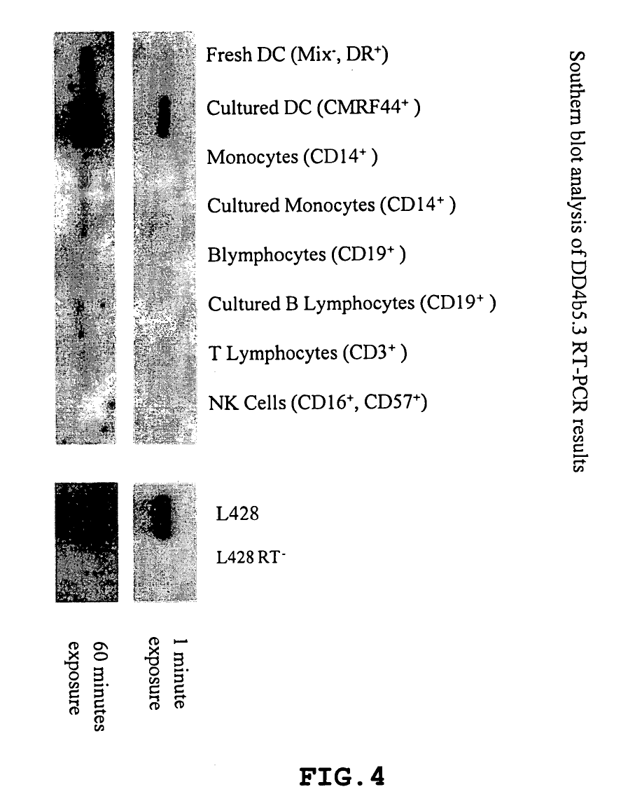

Semi-quantitative RT-PCR was performed on cDNA template derived from normal leukocyte cell populations using specific oligonucleotide primers. The cDNA equivalent from 125 cells was used in each reaction (FIG. 4). To confirm the specificity of the DD4b5.3 RT-PCR (nucleotides 529-944), the products (416 bp) were blotted onto nylon membrane and probed with an internal digoxigenin (Boehringer Manheim) labelled oligonucleotide. Control β2-microglobulin RT-PCR analysis confirmed the template integrity.

Abundant DD4b5.3 message was observed after 1 min exposure, in activated DC (lane 2). and fresh DC (lane 1). Minimal expression in some other leukocyte populations was noted, but only after prolonged 60 min exposure.

iii) DC Lineage

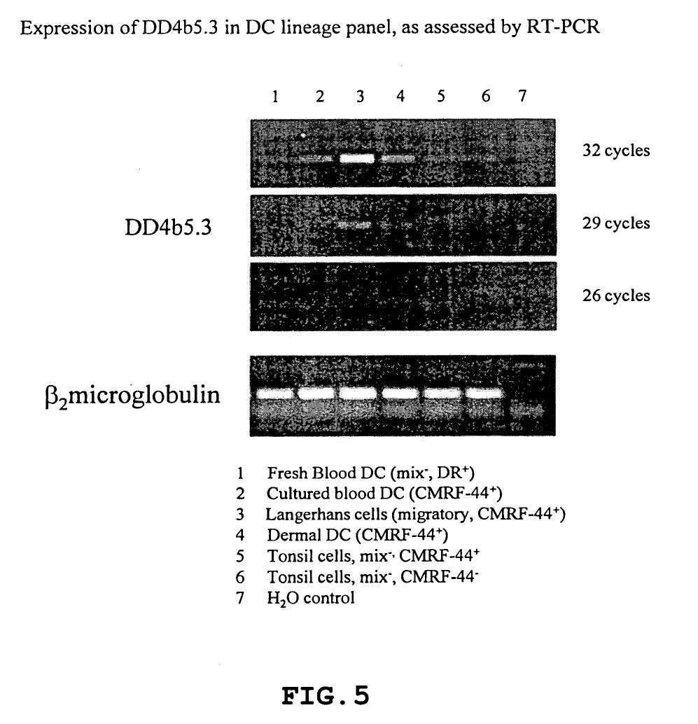

RT-PCR was performed using cDNA template from a range of different DC populations representing a putative DC differentiation/activation pathway. (FIG. 5). Using a range of cycle numbers are estimate of DD4b5.3 mRNA context in each type of DC preparation was obtained, increased levels of DD4b5.3 mRNA were associated with the differentiation/activation of DC derived from the blood and non-lymphoid tissues but little DD4b5.3 mRNA was detected in “mature” tonsil DC.

### CONCLUSION

The enzyme of the invention has a dendritic-cell-restricted expression pattern.

### INDUSTRIAL APPLICATION

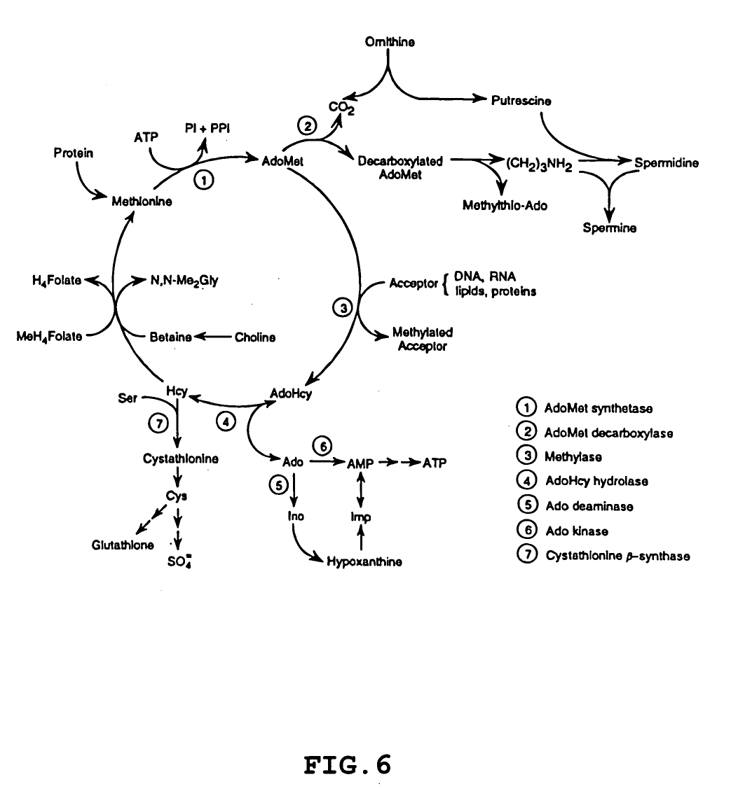

The significant amino acid identity of DD4b5.3 with human AHCY suggests that it has a related enzymatic function (ie. is of the AHCY-type) (see enzyme 4 in FIG. 6). As turnover of (AdoMet) varies between tissues it seems probable that different cell types may express different AHCY like enzymes for the pathways illustrated in FIG. 6 or other pathways. DD4b5.3 may have such a function in DC. Its relatively selective expression in DC and its upregulation at certain stages of DC differentiation/activation suggest it may be critical to DC function.

The novel N terminal sequence of the enzyme may be involved in localising DD4b5.3 to a specific intracellular compartment or play a part in regulating its enzymatic function perhaps by acting as a regulatory domain and binding to other proteins.

The enzyme of the invention will therefore have a number of potential applications. These include in immunomodulation (ie. manipulation of the immune response of a host). The enzyme will also have application in a screening programme to identify substances (usually compounds) which have either a stimulatory or inhibitory effect on its activity. More preferably, the program will be directed to determining the immunosuppressive potential of a test compound with reference to the ability of the compound to inhibit enzyme activity.

In this method, procedures identical or analogous to those outlined in Wolos et al., Journal of Immunology (1993). supra can be employed. Other conventional procedures for screening the effect of a test compound on enzyme activity can also be used.

Compounds identified in this way will have numerous potential applications.

AHCY itself can be inhibited by many nucleosides^{2.6.9}. The compound MDL 28,842 has been reported to be immunosuppressive. It inhibited Con A and PWM induced proliferation of human peripheral blood mononuclear cells¹⁰. MDL 28.8492 inhibited the Con A induced mouse T lymphocyte proliferation but not LPS induced B cell proliferation and reduced T lymphocyte IL-2 and IL-2R production¹⁰. In vivo, MDL 28.842 inhibited the mouse antibody response to the T lymphocyte dependent antigen ovalbumin¹⁰. The same compound prevented collagen induced arthritis in mice¹¹. Administration at 5 mg/kg/day ip for 6 days prolonged skin graft survival to 12.2 days compared to 8.7 days in controls, a superior result to that obtained with Cyclosporin A at 5 mg/kg/day¹². It may also inhibit heart allograft rejection in rats¹².

Equivalent applications are likely for compounds which inhibit the enzyme of the invention.

Blocking or inhibiting the activity of enzyme DD4b5.3 also gives rise to the following potential applications:
Inhibition of DD4b5.3 may block DC function (eg: migration, antigen uptake, antigen processing and presentation or costimulation of T lymphoctyes). This may be profoundly immunosuppressive. DD4b5.3 can be expressed as a protein¹⁷ and nucleoside analogues or other compounds designed which inhibit its function. A precedent for selective functional inhibition is illustrated by the use of the AHCY inhibitory compound C3 ado which inhibits macrophage IL-1 synthesis but not T lymphocyte proliferation¹⁸.
The Identification of safe agents for in vivo use (note similar compounds have been given to animals without major side-effects), which have a selective inhibitory effect on DC may allow the design of an important new immunosuppressive drug. This would have applications in all forms of transplantation and auto-immune disease.
It is (theoretically) possible that blocking DD4b5.3 enzymatic function may have the reverse function, ie: boost their functional capabilities. As such, DD4b5.3 compounds might have inmunostimulatory properties for vaccination eg. against infectious disease or for the immunotherapy of cancer.
It is also conceivable that blocking DD4b5.3 might modify DC growth and differentiation facilitating the collection of DC for therapeutic use.

The enzyme of the invention car, also be used to generate antibodies for example by the method of Kohler and Milstein (Kohler, G, and Milstein, C., Nature 256 495-497 (1975). Due to the restricted expression of the enzyme of the invention, such antibodies will have potential utility in the identification of DC in cytological preparations and tissue sections. The abnormal cells in Hodgkins disease have many features similar to DC. The enzyme is expressed in HD cell lines and the identification of the abnormal cells (Reed Sternberg cells and the related Hodgkins cells) which express the enzyme will be of value in the diagnosis of disorders such as Hodgkins disease²⁰.

The DNA sequence of the invention or any part thereof can also be used as a probe to discriminate for abnormal HD cells by in situ hybridisation. The use of nucleic acid probes is standard, for example as described in Maniatis et al., “Molecular Cloning: A Laboratory Manual”, Cold Spring Harbour (1982).

Other applications include the following:
The DD4b5.3 sequence can be used to design anti-sense (anti-mRNA) reagents to suppress DC function, achieving the possible outcomes described above.
The novel amino acid sequence towards the N terminus of the AHCY-like sequence may be used to target other compounds into the appropriate intracellular site to modify molecular functions, eg: to target other inhibitors of DD4b5.3.
The identification of a novel AHCY-like sequence with additional N terminal sequence suggests there may be an extended family of such AHCY-like enzymes. The cDNA sequence of DD4b5.3 could be used to screen for other members of this family by a range of standard molecular biology techniques.

Those persons skilled in the art will of course appreciate that the above description is provided by way of example only and that the invention is not limited thereto.

### REFERENCES

1. Coulter-Karis D E, Hershfield M S: Sequence of full length cDNA for human S-adenosylhomocysteine hydrolase. Ann Hum Gen 53:169, 1989

2. Chiang P K, Gordon R K. Tal J, Zeng C G. Doctor B P. Pardhasaradhi K. McCann P P: S-Adenosylmethionine and methylation. FASEB 10:471. 1996

3. Borchardt R T: S-adenosyl-L-methionine-dependent macromolecule methyltransferases: potential targets for the design of chemotherapeutic agents. J Med Chem. 23:347, 1980

4. Chiang P K: S-adenosylhomocysteine hydrolase as a pharmacological target for the inhibition of trarsmethylation. Adv Exp Med Biol 165:199, 1984

5. Filgueira L, Zuber M. Juretic A. Luscher U. Caetano V, Harder F, Garotta G. Heberer M. Spagnoli G C: Differential effects of interleukin-2 and CD3 triggering on cytokine gene transcription and secretion in cultured tumor infiltrating lymphocytes. Cell Immunol 150:205, 1993

6. Wnuk S F, Yuan C S, Borchardt R G, Balzarini J. DeClercq E, Robins M J: Nucleic acid related compound.84. Synthesis of 6′-(E and Z)-halohomovinyl derivatives of adenosine, inactivation of S-adenosyl-L-homocysteine hydrolase, and correlation of anticancer and antiviral potencies with enzyme inhibition. J Med Chem 37:3579, 1994

7. Liu S, Wolfe M S, Borchardt R T: Rational approaches to the design of antiviral agents based on S-adenosyl-L-homocysteine hydrolase as a molecular target. Antiviral Research 19:247, 1992

8. Paller A S, Arnsmeier S L, Clark S H, Mirkin B L: Z-4.5′-didehydro-5′-fluoroadenosine (MDL 28.842), an irreversible inhibitor of S-adenosylhomocysteine hydrolase, suppresses proliferation of cultured keratinocytes and squamous carcinoma cell lines (published erratum appears in Cancer Res 1994 Jul. 15;54(14):3952). Cancer Res 53:6058, 1993

9. Schmidt J A, Bomiord R. Gao X. Rhodes J: 3-Deazaadenosine—an inhibitor of interleukin 1 production by human peripheral blood monocytes. Int J Immunopharmacol 12:89, 1990

10. Wolos J A, Frondorf K A, Davis G F, Jarvi E T, McCarthy J R, Bowlin T L: Selective inhibition of T cell activation by an inhibitor of S-adenosyl-L-homocysteine hydrolase. J Immunol 150:3264, 1993

11. Wolos J A, Frondorf K A, Esser R E: immunosuppression mediated by an inhibitor of S-Adenosyl-L-homocysteine hydrolase. J Immunol 151:526, 1993

12. Wolos J A, Frondorf K A, Babcock G F, Stripp S A, Bowlin T L: Immunomodulation by an inhibitor of S-adenosyl-L-homocysteine hydrolase: inhibition of in vitro and in vivo allogeneic responses. Cell Immunol 149:402.1993

13. Yuan C S, Ault-Riche D B, Borchardt R T: Chemical modification and site-directed mutagenesis of cysteine residues in human placental S-adenosylhomocysteine hydrolase. J Biol Chem 271:28009, 1996

14. Ault-Riche D B, Yuan C S, Borchardt R T: A single mutation of lysine 426 of human placental S-adenosylhomocysteine hydrolase inactivates the enzyme. J Biol Chem 269:31472, 1994

15. German D C, Bloch C A, Kredich N M: Measurements of S-adenosylmethionine and L-homocysteine metabolism in cultured human lymphoid cells. Journal of Biological Chemistry 258:10997. 1983

16. Gupta R A, Yuan C. Ault-Riche D B, Borchardt R T: unknown. Arch Biochem Biophys 319:365, 1995

17. Ogawa H, Gomi T, Mueckler M M, Fujioka M. Backlund P S J, Aksamit R R, Unson C G. Cantoni G L: Amino acid sequence of S-adenosyl-L-homocysteine hydrolase from rat liver as derived from the cDNA sequence. Proc Natl Acad Sci USA 84:719. 1987

18. Yuan L, Kuramitsu Y. Li Y, Kobayashi M, Hosokawa M: Restoration of interleukin-2 production in tumor-bearing rats through reducing tumor derived transforming growth factor β by treatment with bleomycin. Cancer Immunol Immunother 41:355, 1995

19. Pike M C, DeMeester C A. Inhibition of phosphoinosotide metabolism in human polymorphonuclear leukocytes by S-adenosylhomocysteine. Journal of Biological Chemistry 263:3592. 1988

20. Sorg U R, Morse T M, Patton W N, Hock B D, Angus H B, Robinson B A, Colls B M, Hart D N J: Hodgkin's cells express CD83, a dendritic cell lineage associated marker. Pathology 29:294. 1997

1. An enzyme having AHCY-type activity which includes amino acids 177 to 314 of the amino acid sequence of FIG. 1 (SEQ ID NO:1), or a functional portion or functional equivalent of said enzyme.

2. An enzyme according to claim 1 which comprises amino acids 183 to 614 of the amino acid sequence of FIG. 1 (SEQ ID NO:1).

3. An enzyme according to claim 1 which comprises amino acids 1 to 614 of the amino acid sequence of FIG. 1 (SEQ ID NO:1).

4. An isolated DNA sequence comprising a nucleotide sequence selected from the group consisting of:
(a) a sequence which encodes an enzyme according to claim 1 or a functional portion or equivalent thereof;
(b) a sequence which is a complement of a sequence (a);
(c) a sequence which is a reverse complement of a sequence (a); and
(d) a sequence which is a reverse sequence of a sequence (a).

5. A DNA sequence according to claim 4 in which sequence (a) comprises nucleotides 529 to 945 of the FIG. 1 (SEQ ID NO:1) sequence.

6. A DNA sequence according to claim 4 in which sequence (a) comprises nucleotides 549 to 1844 of the FIG. 1 sequence.

7. A DNA sequence according to claim 4 in which sequence (a) comprises nucleotides 1 to 1844 of the FIG. 1 (SEQ ID NO:1) sequence.

8. A DNA construct comprising a DNA sequence according claim 4.

9. A DNA construct comprising, in the 5′-3′ direction:
(a) a gene promoter sequence;
(b) an open reading frame coding for at least a functional portion of an enzyme according to claim 1; and
(c) a gene termination sequence.

10. A DNA construct according to claim 9 wherein the open reading frame is in a sense orientation.

11. A DNA construct according to claim 9 wherein the open reading frame is in an anti-sense orientation.

12. A DNA construct comprising, in the 5′-3′ direction:
(a) a gene promoter sequence;
(b) a non-coding region of a gene coding for an enzyme according to claim 1; and
(c) a gene termination sequence.

13. A method for modulating the activity of an enzyme having AHCY-type activity which includes amino acids 177 to 314 of the amino acid sequence of FIG. 1, or a functional portion or functional equivalent of said enzyme in a patient, comprising administering to said patient a DNA construct according to claim 8.

14. A method of amplifying the activity of an enzyme having AHCY-type activity which includes amino acids 177 to 314 of the amino acid sequence of FIG. 1, or a functional portion or functional equivalent of said enzyme in a patient comprising administering to said patient a DNA construct according to claim 10.

15. A method of suppressing the activity of an enzyme having AHCY-type activity which includes amino acids 177 to 314 of the amino acid sequence of FIG. 1, or a functional portion or functional equivalent of said enzyme in a patient comprising administering to said patient a DNA construct according to claim 11.

16. A method of determining the modulatory potential of a compound on an enzyme according to claim 1 which comprises the step of determining the ability of said compound to modulate the activity of said enzyme.

17. An antibody which binds an enzyme according to claim 1.

18. An optionally labeled nucleic acid probe capable of hybridizing, under high stringency, to a nucleotide sequence of FIG. 1 (SEQ ID NO:1).


SEQUENCE LISTING (2 sequences)

SEQ ID NO: 1 - DNA, 2563 bp, Homo sapiens
  CDS (3)..(1847): Open reading frame extends without a stop codon for the full 5′ nucleotide sequence. The initiation codon has yet to be identified.
ggcgcgggcaggtcggagctcggagctgctgcttctggttctcttgtggccaccgtcgctgtccggctgccttgggctgccgaacagacaaggcgtgggccacagcacctcagaagccgacgcagctcgacgcaggggccggcaggagggtgggcgatcgcgtgtcggagggcgccgcgcgggcaggcgggcgggcgccagagggggaaagaggcgggggcggcgggtcagccgctggccgggccggcgggggaatgtcgatgcctgacgcgatgccgctgcccggggtcggggaggagctgaagcaggccaaggagatcgaggacgccgagaagtactccttcatggccaccgtcaccaaggcgcccaagaagcaaatccagtttgctgatgacatgcaggagttcaccaaattccccaccaaaactggccgaagatctttgtctcgctcgatctcacagtcctccactgacagctacagttcagctgcatcctacacagatagctctgatgatgaggtttctccccgagagaagcagcaaaccaactccaagggcagcagcaatttctgtgtgaagaacatcaagcaggcagaatttggacgccgggagattgagattgcagagcaagacatgtctgctctgatttcactcaggaaacgtgctcagggggagaagcccttggctggtgctaaaatagtgggctgtacacacatcacagcccagacagcggtgttgattgagacactctgtgccctgggggctcagtgccgctggtctgcttgtaacatctactcaactcagaatgaagtagctgcagcactggctgaggctggagttgcagtgttcgcttggaagggcgagtcagaagatgacttctggtggtgtattgaccgctgtgtgaacatggatgggtggcaggccaacatgatcctggatgatgggggagacttaacccactgggtttataagaagtatccaaacgtgtttaagaagatccgaggcattgtggaagagagcgtgactggtgttcacaggctgtatcagctctccaaagctgggaagctctgtgttccggccatgaacgtcaatgattctgttaccaaacagaagtttgataacttgtactgctgccgagaatccattttggatggcctgaagaggaccacagatgtgatgtttggtgggaaacaagtggtggtgtgtggctatggtgaggtaggcaagggctgctgtgctgctctcaaagctcttggagcaattgtctacattaccgaaatcgaccccatctgtgctctgcaggcctgcatggatgggttcagggtggtaaagctaaatgaagtcatccggcaagtcgatgtcgtaataacttgcacaggaaataagaatgtagtgacacgggagcacttggatcgcatgaaaaacagttgtatcgtatgcaatatgggccactccaacacagaaatcgatgtgaccagcctccgcactccggagctgacgtgggagcgagtacgttctcaggtggaccatgtcatctggccagatggcaaacgagttgtcctcctggcagagggtcgtctactcaatttgagctgctccacagttcccacctttgttctgtccatcacagccacaacacaggctttggcactgatagaactctataatgcacccgaggggcgatacaagcaggatgtgtacttgcttcctaagaaaatggatgaatacgttgccagcttgcatctgccatcatttgatgcccaccttacagagctgacagatgaccaagcaaaatatctgggactcaacaaaaatgggccattcaaacctaattattacagatactaatggaccatactaccaaggaccagtccacctgaaccacacactctaaagaaatattttttaagataacttttattttcttcttactcctttcctcttgatttttttcctataatttcattcttgttttttcatctcattatccaagttctgcagaccacacaggaacttgcttcatggctctttagatgaaatagaagttcagggtccctcactctagtcactaaagaaggattttactcccccagcccagaaaggtgattcttctctttaccatttctggggactttagtcttaattaggtaccttattaacaggaaatgctaaggtaccttctctgtggaacaatctgcaatgtctaaatcgccttaaaagagcccatttcttagctgctgaaatcagtgctctttcacttcttcagagaagcagggatggtacctacccggcaggtaggttagatgtgggtggtgcatgttaatttcccttagaagttccaagccctgtttcctgcgtaaaggtggtatgtccagttcagagatgtgtataatgagcatggcttgttaagatcaggaggcccacttggatttatagtatagcccttcctccactcccaccagacttgctcatttttcgagtttttaactagactacactctatttgagtttaattttgtcctctaggatttatttctgttgtccaaaaaaaaaaaaaaaaaaaa

SEQ ID NO: 2 - protein, 614 aa, Homo sapiens
RGQVGARSCCFWFSCGHRRCPAALGCRTDKAWATAPQKPTQLDAGAGRRVGDRVSEGAARAGGRAPEGERGGGGGSAAGRAGGGMSMPDAMPLPGVGEELKQAKEIEDAEKYSFMATVTKAPKKQIQFADDMQEFTKFPTKTGRRSLSRSISQSSTDSYSSAASYTDSSDDEVSPREKQQTNSKGSSNFCVKNIKQAEFGRREIEIAEQDMSALISLRKRAQGEKPLAGAKIVGCTHITAQTAVLIETLCALGAQCRWSACNIYSTQNEVAAALAEAGVAVFAWKGESEDDFWWCIDRCVNMDGWQANMILDDGGDLTHWVYKKYPNVFKKIRGIVEESVTGVHRLYQLSKAGKLCVPAMNVNDSVTKQKFDNLYCCRESILDGLKRTTDVMFGGKQVVVCGYGEVGKGCCAALKALGAIVYITEIDPICALQACMDGFRVVKLNEVIRQVDVVITCTGNKNVVTREHLDRMKNSCIVCNMGHSNTEIDVTSLRTPELTWERVRSQVDHVIWPDGKRVVLLAEGRLLNLSCSTVPTFVLSITATTQALALIELYNAPEGRYKQDVYLLPKKMDEYVASLHLPSFDAHLTELTDDQAKYLGLNKNGPFKPNYYRY